(19) United States
(12) Patent Application Publication
(10) Pub. No.: US 2022/0215553 A1
(43) Pub. Date: Jul. 7, 2022

(54) DEEP LEARNING-BASED SEGMENTATION OF CORNEAL NERVE FIBER IMAGES

(71) Applicants: Voxeleron, LLC, San Francisco, CA (US); THE JOHNS HOPKINS UNIVERSITY, Baltimore, MD (US)

(72) Inventors: Jonathan D. Oakley, Pleasanton, CA (US); Daniel B. Russakoff, Pleasanton, CA (US); Joseph L. Mankowski, Severna Park, MD (US)

(73) Assignees: Voxeleron, LLC, San Francisco, CA (US); THE JOHNS HOPKINS UNIVERSITY, Baltimore, MD (US)

(21) Appl. No.: 17/612,104

(22) PCT Filed: May 18, 2020
(86) PCT No.: PCT/US2020/033425

§ 371 Date: Nov. 17, 2021

Related U.S. Application Data
(60) Provisional application No. 62/849,356, filed on May 17, 2019.

(51) Int. Cl. G06T 7/00; G06T 7/11; G16H 30/40; G16H 50/20; G06T 7/80; G06N 3/08

(52) U.S. Cl. CPC G06T 7/0014; G06T 7/11; G16H 30/40; G16H 50/20; G06T 2207/10056; G06N 3/088; G06T 2207/30041; G06T 2207/20084; G06T 7/80

(57) ABSTRACT

This disclosure relates to a method for automating segmentation of corneal nerve fibers based on a deep learning approach to segmentation. Methods of the invention offer more robust results by utilizing the power of supervised learning methods in concert with the pre- and post processing techniques documented.

### CROSS-REFERENCE TO RELATED APPLICATIONS

This application claims priority to U.S. Provisional Application No. 62/849356, filed on May 17, 2019, the contents of which are incorporated by reference.

### FIELD OF THE INVENTION

This disclosure relates generally to medical devices. More particularly, this disclosure relates to automated methods for segmenting corneal nerve fiber images.

### BACKGROUND

Peripheral neuropathy is a frequent neurological complication occurring in a variety of pathologies including diabetes, human immunodeficiency virus (HIV), Parkinson's, multiple sclerosis, as well as a number of systemic illnesses. In HIV, for example, it affects more than one-third of infected persons, with the typical clinical presentation is known as distal sensory polyneuropathy, a neuropathy that is characterized by bilateral aching, painful numbness or burning, particularly in the lower extremities. This debilitating disorder greatly compromises patient quality of life.

Conventionally, monitoring patients for peripheral neuropathies is performed by skin biopsy. The skin biopsy is used to measure loss of small, unmyelinated C fibers in the epidermis—one of the earliest detectable signs of damage to the peripheral never system. However, skin biopsy is a painful and invasive procedure, and longitudinal assessment requires repeated surgical biopsies. The development and implementation of non-invasive approaches is therefore paramount.

One promising non-invasive approach for detecting peripheral neuropathies is with corneal nerve assessments. Such assessments may be made by analyzing images of nerve fibers. Unfortunately, such methods are poorly developed and lack the accuracy needed to diagnosis and monitor patients.

### SUMMARY

This disclosure relates to systems and methods for assessing corneal nerve fibers from images captured by non-invasive imaging techniques to generate data for detecting neural pathologies. In particular, methods of the invention take images of corneal nerve fibers, pre-process those images, apply a deep learning based segmentation to the data, and report nerve fiber parameters, such as length, density, tortuosity, etc. Such metrics are useful clinically to diagnosis and stage a variety of neuropathies that attack the central nervous system. The image data may be from different modalities, including in vivo confocal microscopy, optical coherence tomography and other sensing techniques that create images where the nerves are visible in 2 or 3 dimensional data.

In one aspect, the invention provides a method that includes obtaining imaging data comprising images of nerve fibers; pre-processing the imaging data; training a classifier to recognize nerve fiber locations in the images using pre-processed images and labels, e.g., hand drawn labels; applying the trained classifier to assign a score to each of a plurality of image pixels of an input image, wherein the score comprises a probability, or represents a likelihood, that each of the plurality of image pixels represents a nerve fiber; and post-processing the input image to create a new image (e.g., a binary image) that indicates locations of pixels that represent nerves from the input image.

In some embodiments, pre-processing the imaging data comprises equalizing contrast and correcting non-uniform illumination specific to each of the images of never fibers. Equalizing may be performed using at least one of a top-hat filter, low-pass filtering and subtraction, or flat-fielding based on a calibration step.

In some embodiments, the imaging data comprises images of never fibers that are taken with a microscope. For example, the imaging data may comprise images taken with a confocal microscope, e.g., by in vivo corneal confocal microscopy. In other embodiments, the imaging data comprises optical coherence tomography data. In such instances where optical coherence tomography data is used, contrast equalization by methods of the invention may be based on limiting the integration range, or based on one of a minimum, a maximum, an average, a sum, or a median in the depth direction.

Some steps of the method are preferably performed offline. For example, training the classifier may be performed offline. Preferably, applying the trained classifier to assign scores to image pixels is performed online.

In some embodiments, methods of the invention employ one of a classifier, a detector, or a segmenter that comprises one of a deep neural network or a deep convolutional neural network. The deep neural network may comprise an encoding and decoding path, as in the auto-encoder architecture, or for example, a SegNet or U-net architectures.

In some embodiments, post-processing comprises thresholding the input image and a skeletonization of the thresholded image. Post-processing may comprise a classifier trained to take a probability image and return a binary image. Alternatively, post-processing may comprise a thresholding of the input image and a center-line extraction of the thresholded image. The binary image may be useful for diagnosing neuropathies. The binary image may be useful for monitoring a patient response to a treatment, e.g., a chemotherapy treatment.

In other aspects, the present disclosure relates to a non-transitory computer-readable medium storing software code representing instructions that when executed by a computing system cause the computing system to perform a method of identifying the nerve fibers in an image. The method comprises obtaining an imaging data set containing an image of nerve fibers; preprocessing the data to equalize the contrast and correct non-uniform illumination specific to each of the images; training, preferably, offline, a segmenter or classifier to recognize nerve locations in an image using the preprocessed images as input and hand drawn labels as truth; applying, preferably online, the trained classifier to assign a probability of representing a nerve to each of the image pixels of an input image; and post-processing the probability image to create a binary image indicating the locations of all of the pixels in the input image representing nerve fibers.

Preferably, the contrast equalization step comprises one of a top-hat filter, a low-pass filtering and subtraction step, or flat-fielding based on a calibration step. The image data may comprise images from a microscope, such as a confocal microscope. Alternatively, the image data comprise optical coherence tomography data. In embodiments where the image data comprises optical coherence tomography data, the contrast equalization step used for the optical coherence tomography data may be based on limiting the integration range, or based on minimum or maximum or average or sum or median in the depth direction.

In preferred embodiments, the classifier used by methods of the invention comprise one of a deep neural network or a deep convolutional neural network. The deep convolutional neural network may comprise an auto-encoder architecture. The auto-encoder architecture may follow a SegNet architecture or a U-net architecture.

In certain embodiments, post-processing comprises thresholding of the image and a skeletonization of the thresholded image. In some embodiments, post-processing comprises a classifier trained to take a probability image and return a binary image. In other embodiments, the post-processing involves thresholding of an image and a center-line extraction of the thresholded image or involves using a classifier trained to take a probability image and return a binary image.

### BRIEF DESCRIPTION OF THE DRAWINGS

FIG. 1 shows a high-level illustration of a work pipeline according to aspects of the invention.

FIG. 2 shows an exemplary contrast-equalization pipeline.

FIG. 3 shows a data segmentation technique according to aspects of the invention.

FIG. 4 illustrates application of a trained network.

FIG. 5 shows a schematic of a U-Net architecture that is used to learn and then segment

FIG. 6 illustrates a post-processing pipeline according to aspects of the invention.

### DETAILED DESCRIPTION

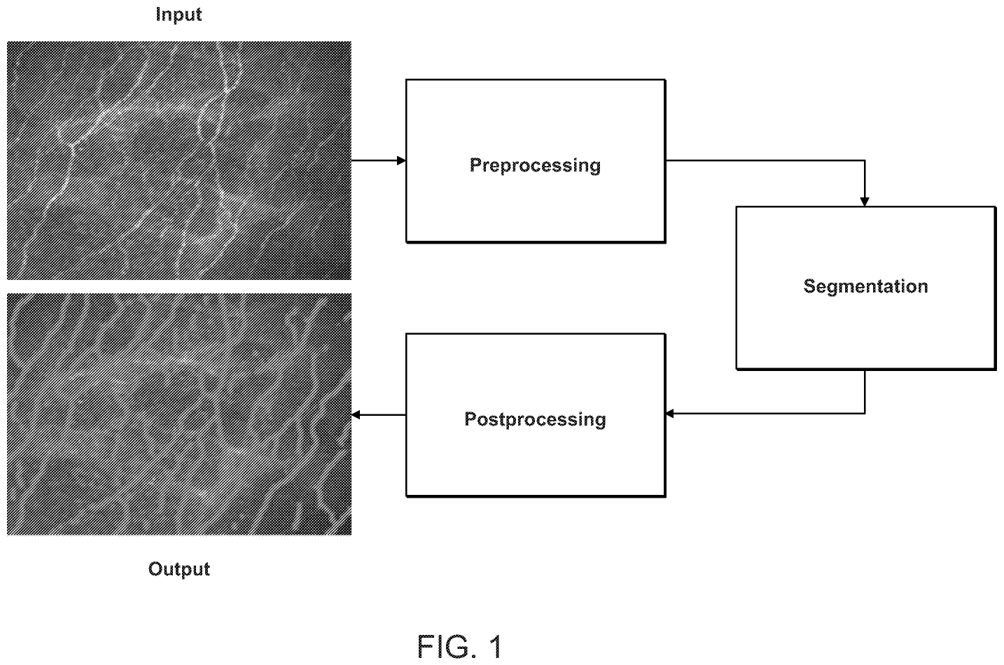

This disclosure provides systems and methods for robust, repeatable, quantification of corneal nerve fibers from image data. The cornea is the most densely innervated tissue in the body and analysis of corneal nerve is sensitive for detecting small sensory nerve fiber damage. Segmentation of the nerve fibers in these images is a necessary first step to quantifying how the corneal nerve fibers may have changed as a result of disease or some other abnormality. The procedure, at a high level is detailed in FIG. 1 and explained in more throughout this disclosure. To briefly describe the method as illustrated in FIG. 1, methods of the invention take in input image data in which the corneal nerve fibers can be visualized, pre-process that image data, apply a deep learning based segmentation to the data, and report nerve fiber parameters, such as length, thickness, density, tortuosity, etc. Such metrics can be used clinically to detect and stage a variety of neuropathies that attack the central nervous system. The image data may be from different modalities, including confocal microscopy, optical coherence tomography, and other sensing techniques that create images wherein the nerves are visible. Image data may be in any form including 2-dimensional or 3-dimensional image data.

FIG. 1 shows a high-level illustration of a work pipeline according to aspects of the invention. An exemplary input image showing never fibers is shown. The input image undergoes at least three independent steps: pre-processing; segmentation; and post-processing. Each step is described in turn below. An exemplary output image is depicted underneath the input image.

### Pre-Processing

Acquired image data may be messy or may come from different sources. To feed them into machine learning systems or neural network according to methods of the invention, the data may need to be standardized and/or cleaned up. Preprocessing may be used to reduce training complexity—i.e., by narrowing the learning space—and/or to increase the accuracy of applied algorithms, e.g., algorithms involved in image segmentation. Data preprocessing techniques according to aspects of the invention might include may comprise one of an intensity adjustment step, or a contrast equalization step. Additionally, pre-processing may include converting color images to grayscale to reduce computation complexity. Grayscale is generally sufficient for recognizing certain objects.

Pre-processing may involve standardizing images. One important constraint that may exist in some machine learning algorithms, such as convolutional neural networks, is the need to resize the images in the image dataset to a unified dimension. For example, the images may be scaled to a specific width and height before being fed to the learning algorithm.

Pre-processing may involve techniques for augmenting the existing dataset with perturbed versions of the existing images. Scaling, rotations and other affine transformations may be involved. This may be performed to enlarge a dataset and expose a neural network to a wide variety of variations of images. Data augmentation may be used to increase a probability that the system recognizes objects when they appear in any form and shape. Many preprocessing techniques may be used to prepare images for train a machine learning model. In some instances, it may be desirable to remove variant background intensities from images to create a more uniform appearance and contrast. In other instances, it may be desirable to brighten or darken your images. Preferably, pre-processing comprises an intensity adjustment step, or contrast equalization step.

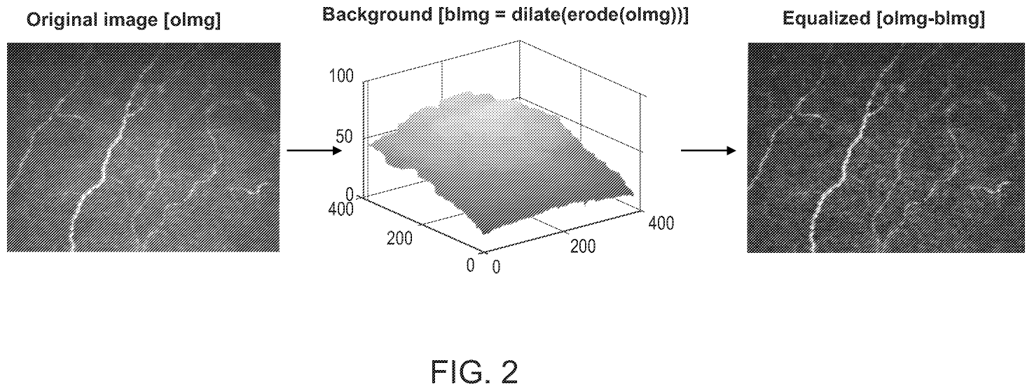

FIG. 2 shows an exemplary contrast-equalization pipeline. This contrast-equalization provides a method for equalizing contrast across the image to support segmentation. Contrast- equalization is a computer vision technique that supports segmentation by, for example, accounting for inhomogeneous intensity distribution across the image. This ensures that pixels representing the foreground (brighter nerve pixels) and those of the background (darker surrounding tissue pixels) are more uniformly distributed. This step may reduce variance in the training set ahead of the segmentation step. The means by which preprocessing images may occur is via a top-hat filter. A top-hat filter is mathematically equivalent to performing a morphological opening operation (an erosion followed by a dilation) and then subtracting that result from the original. The effect of this is to model the background of the image (ignoring the foreground) and then subtracting that background to flatten the image so that all background pixels have more or less the same intensity. The top-hat filter is just one example of such a contrast-equalization approach. Alternatives include, but are not limited to: simply smoothing the image data to get a low frequency image that describes the background, then dividing the input image by the low frequency image to more uniformly correct overall brightness. Alternatively, it may be useful to instead fit a surface to the image data and create the same adjustment.

In some embodiments, an explicit calibration step may be used in instances where the inhomogeneity results mostly from the optics of the system. This is often referred to as flat fielding, and involves imaging a uniform target—such as a white, flat, surface—to directly measure how intensity falls off at the periphery. The correction is then applied based on this calibration image.

In other embodiments, a simple histogram equalization or adaptive histogram equalization step may be employed. A person of skill in the art will recognize that any technique that can more evenly distribute the intensities of the foreground and background pixels may be useful for pre-processing step. This of course may depend on the modality. For example, in optical coherence tomography data, the process might involve restricting the integration range of the data used to create a 2-dimensional image from a 3-dimensional image, as optical coherence tomography data is depth resolved. In such cases, a 3-dimensional volume, acquired at the cornea, may be converted to 2-dimensional via integration of the data through an axial direction. Alternatively, the 2-dimensional image may be produced by taking the maximum, minimum, median or average value through the axial dimension. Furthermore, the choice of axial range could be limited based on structural landmarks. Once the pre-processing is complete, the equalized images may then be provided to a segmentation module.

### Segmentation

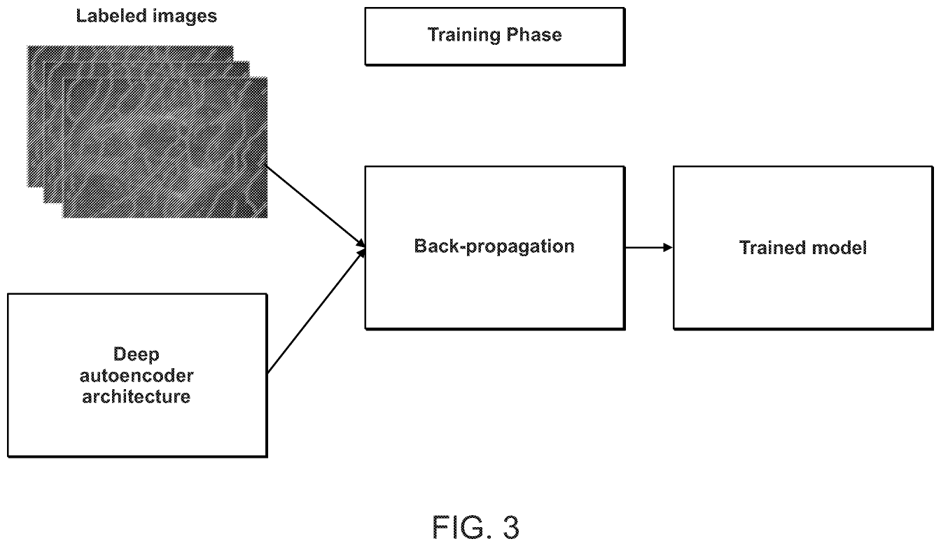

Methods of the invention provide for the automated segmentation of fibers. Such methods provide for a more accurate and repeatable measure of the nerve fiber density and calculation of higher order features from the segmentations such as tortuosity, curvature statistics, branch points, bifurcations etc. This ability to automatically and accurately quantify never fibers from image data is useful for diagnosing neuropathies secondary to a very large number of pathologies, including diabetes and HIV. It can also detect and monitor neuropathies stemming from chemotherapy and other potentially damaging treatment protocols. An exemplary segmentation pipeline is depicted in FIGS. 3 & 4.

FIG. 3 shows a data segmentation technique according to aspects of the invention. Preferably, this technique is done using back-propagation to learn the weights of the network.

Segmentation, according to aspects of the invention, may rely on a classifier. The classifier offers a supervised learning approach in which a computer program learns from input data, e.g., images with hand labeled nerves, and then uses this learning to classify new observations, e.g., locations of nerves from unlabeled images. The classifier may comprise any known algorithm used in the art. For example, the classifier may comprise a linear classifier, logistic regression, naive bayes classifier, nearest neighbor, support vector machines, decision trees, boosted trees, random forest, or a neural network algorithm. Preferably, the classifier uses a deep convolutional neural network, for example, as described in, Ronneberger, 2015, U-Net: Convolutional Networks for Biomedical Image Segmentation, incorporated by reference. Alternative architectures may include an auto-encoder, such as the auto-encoder described in Badrinarayanan, 2015, SegNet: A Deep Convolutional Encoder-Decoder Architecture for Robust Semantic Pixel-Wise Labelling, incorporated by reference. Preferably, the segmentation is performed using a deep convolutional neural network U-Net as a classifier for associating each input pixel with a probability of being a nerve pixel. Alternative embodiments include, but are not limited to any supervised learning based classifier including: support vector machine, a random forest, a Deep conventional neural network auto encoder architecture, a Deep convolutional V-Net architecture (3d U-Net), or a logistic regression. The model is trained using input images which have had the nerves hand-labeled to serve as a ground truth (See, FIG. 3). Hand-labeling may be performed using a computer program to label, or mark, locations of nerves. This training may take place offline, i.e., without an internet connection. In some embodiments, segmentation may involve dividing the images into patches and analyzing the fibers in each patch, for example, as described in U.S. Pat. No. 9,757,022, which is incorporated by reference. Training results in a trained model suitable for taking new corneal images and generating predictions as to the locations of their nerves. It may also be simply a score, an intensity response to the processing where the higher the number the more likely the pixel is a nerve.

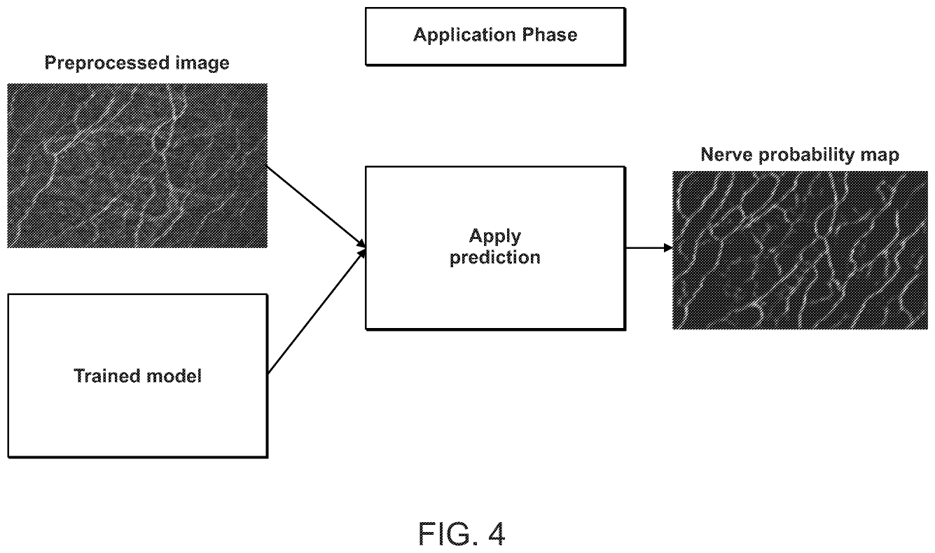

FIG. 4 illustrates application of a trained network. In particular, once the training of the network is complete, the network may be applied in an application phase wherein the image is presented and passed through the network to produce an output probability map of the nerves. At this stage the network's weights may be fixed and the data may be passed through the layers of the network. The output may comprise a probability map assigning a probability (e.g., pij value) to each pixel where pij represents the probability that pixel (i.j) represents a nerve. This probability map may then sent be provided to a post-processing module where it is turned into a binary map where each “on” pixel represents a nerve.

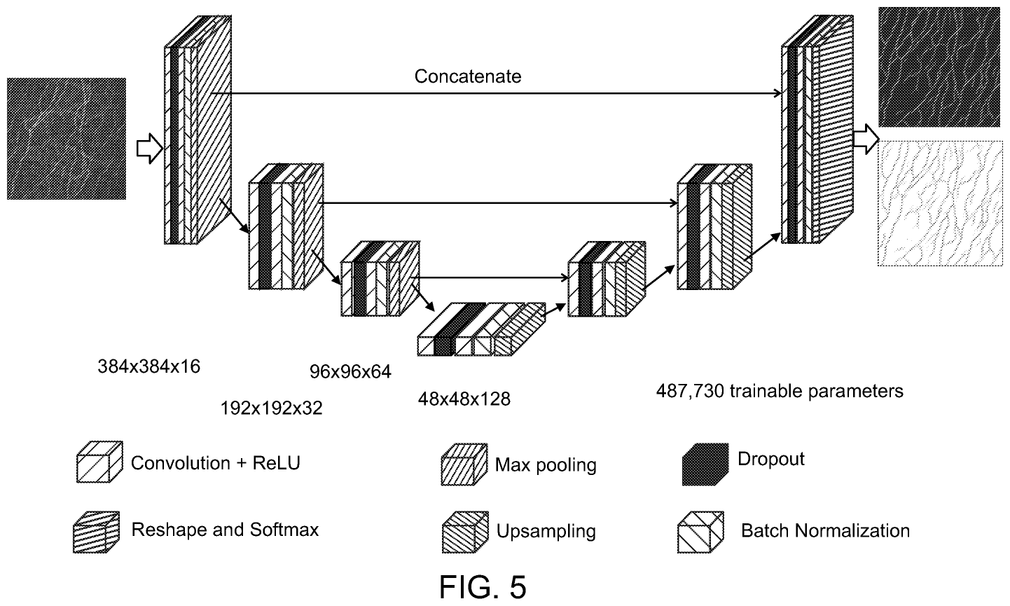

FIG. 5 shows a schematic of a U-Net architecture that is used to learn and then segment the nerve fibers in the image data. The example data shown is from a confocal microscope.

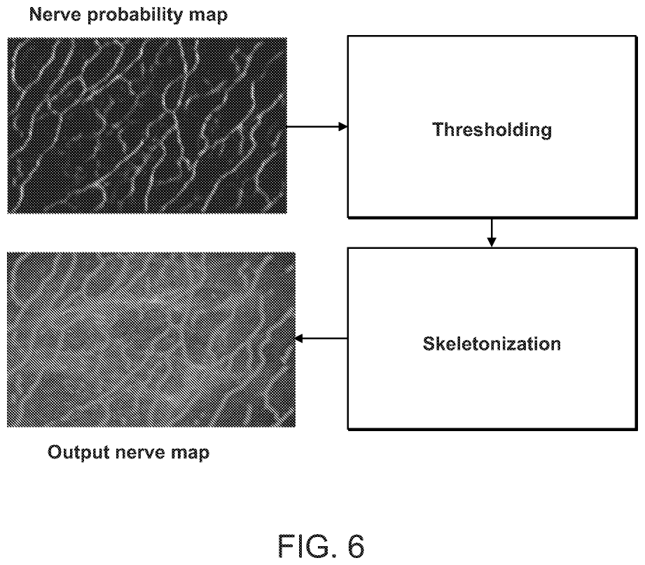

FIG. 6 illustrates a post-processing pipeline according to aspects of the invention. The deep learning based segmentation outputs a probability map of nerves that is post-processed. In post-processing, the probability may thresholded to produce a binary map. This may be performed with thresholding methods, and then a binarization. An optional step of skeletonization may be applied in order to more easily support automating the counting nerve fiber lengths.

Post-processing may involve two steps: thresholding and skeletonization. For example, first the probability map may be thresholded to separate the foreground (nerve pixels) from the background. Preferably this is performed using a method referred to as Otsu's method. Otsu's method, named after Nobuyuki Otsu, performs automatic image thresholding. In the simplest form, the algorithm returns a single intensity threshold that separate pixels into two classes, foreground and background. This threshold is determined by minimizing intra-class intensity variance, or equivalently, by maximizing inter-class variance. Otsu's method is a one- dimensional discrete analog of Fisher's Discriminant Analysis, is related to Jenks optimization method, and is equivalent to a globally optimal k-means performed on the intensity histogram. The extension to multi-level thresholding was described in the original paper, and computationally efficient implementations have since been proposed. For example, as described in, Nobuyuki Otsu (1979), A threshold selection method from gray-level histogram, IEEE Trans Sys Man Cyber, 9 (1): 62-66, incorporated by reference. Although, a number of alternative methods may be used including: a non-maximum suppression followed by hysteresis thresholding, a k-means clustering, a spectral clustering, a graph cuts or graph traversal, or level sets.

Optionally, a skeletonization step may be applied. Thresholding provides a good estimate of the number of nerve pixels. What may be desired, however, is a count of the number of nerves and their lengths. If a person simply counted the number of pixels from the thresholded image, one may overcount images with thicker nerves and score lengths incorrectly. It may also help as an important step ahead of deriving higher order features such as curvature and tortuosity that are useful clinically. Thus is may be preferable to use an “skeletonization” algorithm to reduce the width of the thresholded nerves to 1 pixel. For example, as described in Shapiro, 1992, Computer and Robot Vision, Volume I. Boston: Addison-Wesley. Other methods may include: a center line extraction, which finds the shortest path between two extremal points, medial axis transform, ridge detection, grassfire transform. Skeletonization, according to methods of the invention, is optional as one might want to also measure nerve fiber width as a clinical end point. Accordingly, it may be desirable to not skeletonize the data if, for example, nerve fiber width is an important parameter. The output of post-processing is a binary image where each “on” pixel represents a segmented nerve.

The binary image may be used for analyzing and quantifying nerve fibers. For example, as described in Al-Fandawi, 2016, A fully automatic nerve segmentation and morphometric parameter quantification system for early diagnosis of diabetic neuropathy in corneal images. Comput Methods Programs Biomed;135:151-166; Annunziata, 2016, A fully automated tortuosity quantification system with application to corneal nerve fibres in confocal microscopy images, Medical image analysis, 32:216-232; Chen X, 2017, An Automatic Tool for Quantification of Nerve Fibers in Corneal Confocal Microscopy Images, IEEE Trans Biomed Eng, 64:786-794; Dorsey J L, 2015, Persistent Peripheral Nervous System Damage in Simian Immunodeficiency Virus-Infected Macaques Receiving Antiretroviral Therapy, Journal of neuropathology and experimental neurology, 74:1053-1060; Dorsey, 2014, Loss of corneal sensory nerve fibers in SIV-infected macaques: an alternate approach to investigate HIV-induced PNS damage. The American journal of pathology 184:1652-1659, Dabbah, 2010, Dual-model automatic detection of nerve-fibres in corneal confocal microscopy images, Medical Image Computing and Computer-Assisted Intervention—MICCAI, 300-307, Oakley, 2018, Automated Analysis of In Vivo Confocal Microscopy Corneal Images Using Deep Learning, ARVO Meeting Abstracts, Laast V A, 2007, Pathogenesis of simian immunodeficiency virus-induced alterations in macaque trigeminal ganglia, Journal of neuropathology and experimental neurology, 66:26-34, Laast V A, 2011, Macrophage-mediated dorsal root ganglion damage precedes altered nerve conduction in SIV-infected macaques, The American journal of pathology, 179:2337-2345, Mangus L M, Unraveling the pathogenesis of HIV peripheral neuropathy: insights from a simian immunodeficiency virus macaque model, ILAR, 54:296-303, each of which is incorporated herein by reference.

What is claimed is:

1. A method comprising:
obtaining imaging data comprising images of nerve fibers;
pre-processing the imaging data;
training a classifier to recognize nerve fiber locations in the images using the pre-processed images and labels;
applying the trained classifier to assign a score to each of a plurality of image pixels of an input image, wherein the score represents a likelihood that each of the plurality of image pixels represents a nerve fiber; and
post-processing the input image to create a new image that indicates locations of pixels that represent nerves in the input image.

2. The method of claim 1, wherein pre-processing the imaging data comprises equalizing contrast and correcting non-uniform illumination specific to each of the images of never fibers.

3. The method of claim 2, wherein equalizing is performed using at least one of a top-hat filter, low-pass filtering and subtraction, or flat-fielding based on a calibration step.

4. The method of claim 3, wherein the imaging data comprises images of never fibers that are taken with a microscope.

5. The method of claim 4, wherein the microscope is a confocal microscope.

6. The method of claim 1, wherein the imaging data comprises optical coherence tomography data.

7. The method of claim 2, wherein the contrast equalization used for the data is based on limiting the integration range, or based on one of a minimum, a maximum, an average, a sum, or a median in the depth direction.

8. The method of claim 1, wherein the step of training a classifier is performed offline, and the step of applying the trained classifier is performed online.

9. The method of claim 1, wherein the labels comprise hand drawn labels.

10. The method of claim 1, wherein the classifier comprises one of a deep neural network or a deep convolutional neural network.

11. The method of claim 10, wherein the deep convolutional neural network comprises an encoding and decoding path.

12. The method of claim 11, wherein the deep convolutional neural network comprises an auto-encoder architecture.

13. The method of claim 12, wherein the auto-encoder architecture comprises one of a SegNet architecture or a U-net architecture.

14. The method of claim 1, wherein post-processing comprises thresholding a result of the trained classifier.

15. The method of claim 1, wherein post-processing comprises a thresholding of the input image and a skeletonization of the thresholded image.

16. The method of claim 1, wherein post-processing comprises a classifier trained to take one of a probability image or a likelihood image and return a binary image.

17. The method of claim 1, wherein post-processing comprises thresholding of the input image and a center-line extraction of the thresholded image.

18. The method of claim 1, wherein post-processing comprises a classifier trained to take a probability image and return a binary image.

19. The method of claim 1, wherein the new image is useful for diagnosing neuropathies or for monitoring a patient response to a treatment.

20. The method of claim 1, wherein new image is further analyzed for parameters such as never fiber length, length density, never count, branching, bifurcations, or tortuosity.
